(19) United States
(12) Patent Application Publication
(10) Pub. No.: US 2002/0168642 A1
(43) Pub. Date: Nov. 14, 2002

(54) Sequencing duplex DNA by mass spectroscopy

(76) Inventor: Andrzej Drukier, Herndon, VA (US)

Correspondence Address: VENABLE, Post Office Box 34385, Washington, DC, 20043-9998, US

(21) Appl. No.: 09/816,673

(22) Filed: Mar. 26, 2001

Related U.S. Application Data
(63) Continuation of application No. PCT/US98/19946, filed on Sep. 24, 1998.
Application No. PCT/US98/19946 is a continuation-in-part of application No. 08/254,761, filed on Jun. 6, 1994, now abandoned.

(51) Int. Cl. C12Q 1/68; C07H 21/04

(52) U.S. Cl. 435/6; 534/15; 536/23.1

(57) ABSTRACT

For the determination of masses of macromolecular analytes with particular application to DNA sequencing by mass spectroscopy, novel strategies of sample preparation and labeling decrease macromolecule breakage, improve identification of population members, aid attainment of a single charge state for the heterogeneous analyte inputs, and increase the sensitivity of detection of the fractionated macromolecules.

### BACKGROUND OF THE INVENTION

The invention relates to determining masses of macromolecular analytes by mass spectroscopy and is suitable for sequencing duplex DNA. More specifically, the invention provides methods of sample preparation and labeling to decrease macromolecule breakage, improve identification of population members, aid attainment of a single charge state for heterogeneous analyte inputs, and increase the sensitivity of detection of the fractionated macromolecules.

In the Maxam-Gilbert or Sanger sequencing strategies, a DNA to be sequenced is processed to generate four representative populations of single stranded fragments. All population members have one common end and the other end is of chosen variability. For a single population, the variable ends terminate at one of the four bases: A, T, G or C (see Table 1 for nomenclature). All possible termini of the chosen base are represented within a particular population. For brevity herein, such populations are generically designated Pop. There are four Pops for each nucleic acid to be sequenced. Fractionations of each of the four Pop are performed to order DNA fragments by size, generating bands of fragments. Data from the four orderings are compared to identify consecutively the bands representing successively longer fragments. The sequence of A, T, G and C subunits is read beginning from the common end, until the capacity to resolve adjacent bands is lost. To assemble longer runs of sequence, individual reads are recognized by their overlaps, aligned and merged.

TABLE 1
DNA subunits, symbols and masses.

| phosphorylated subunits | symbol | mass (amu)* |
|---|---|---|
| deoxyguanidine-OP(OH)₂O— | G | 329.2 |
| deoxyadenosine-OP(OH)₂O— | A | 313.2 |
| deoxycytidine-OP(OH)₂O— | C | 289.2 |
| thymidine-OP(OH)₂O— | T | 304.2 |

*The subunits within DNA lack one H₂O as compared to the free subunits.

Currently the fractionation process most employed for resolving Pop members is gel electrophoresis, in which smaller fragments move faster through the sieving gel matrix. In the relevant size range of several hundred bases, much higher spatial resolution is achieved in gels with single stranded DNAs rather than duplex DNAs. Thus single stranded DNAs have been preferred for size ordering. In preparation for gel electrophoretic separations, product and template strands are separated by combinations of high pH, treatment with denaturants and/or heating which disrupt the hydrogen bonds between template and newly polymerized strands.

The capacity to accurately resolve successive fragment bands begins to deteriorate at about 400-500 base lengths, with rare gel fractionations yielding useful data out to 1000 subunits, which is equivalent to a mass of about 300,000 amu. One of the factors which limits the length of sequence reads is the limited predictability in the positions of successive bands. In general, longer strands have less gel electrophoretic mobility.

Brennan, U.S. Pat. No. 5,174,962, and Mills, U.S. Pat. No. 5,221,518 describe sequencing single stranded populations of nucleic acid fragments (DNA or RNA) by separating using PAGE and then transferring to a mass spectrophotometer. Brennan combusts the intermediates before mass spectrometry and Mills uses a mass spectrometer to measure the relative abundance of components by mass.

In Levis et al., U.S. Pat. No. 5,580,733, a mass spectroscopy sequencing method uses single-stranded molecules of 17 bases with a light-absorbing matrix. Scission occurred with molecules 65 bases long.

Likewise, Köster, U.S. Pat. No. 5,547,835, relates to sequencing single-stranded DNA using mass spectroscopy. The sequencing reaction is performed using a template bound to a solid support and cleaving the product from the solid support before mass spectroscopy.

In Köster, U.S. Pat. No. 5,605,798, a method of determining whether a specific mutation is present in a short fragment of DNA uses mass spectrometry to measure the difference in mass a single base pair substitution confers compared to the wild type allele. The mass of one or a few DNA molecules of the same length is measured, not the mass of a large population of molecules that differ in length and mass. Williams et al., “Time-of Flight Mass Spectrometry of Nucleic Acids by Laser Ablation and Ionization from a Frozen Aqueous Matrix,” Rapid Communications in Mass Spectrometry 4: 348-351 (1990) describes sequencing a DNA molecule of 28 base pairs.

MS systems for DNA analysis must provide information over a broad mass range corresponding to DNAs ten to thousands of subunits long. Two systems that have been suggested are Fourier Transform Ion Cyclotron Resonance (FT-ICR) MS and time of flight (TOF) MS systems. Each system has benefits and problems.

With FT-ICR, a homogenous magnetic field maintains analyte ions in orbits (“High-resolution accurate mass measurements of biomolecules using a new electrospray ionization ion cyclotron resonance mass spectrometer,” Winger, Brian E. et al.; J. Am. Soc. Mass Spectrom., 4(7), 566-77, 1993). The orbital frequency is proportional to the charge/mass (q/m) ratio, and the quantities determined by Fourier transform deconvolution of the ICR signal output. For FT-ICR systems with strong homogenous fields maintained by superconducting magnets, even single orbiting molecules can be detected. Masses above 100,000 amu have been determined.

Electrospray ionization (ESI) is a compatible, relatively gentle ionization methodology (“Selected-ion accumulation from an external electrospray ionization source with a Fourier-transform ion cyclotron resonance mass spectrometer,” Bruce, James E. et al.; Rapid Commun. Mass Spectrom., 7(10), 914-19, 1993). Electrons are sprayed onto vaporizing droplets and macromolecules retain charge as the water evaporates. The hydrogen bond supported, duplex structure of input DNAs can be retained in vacuum provided that the negative charges of the phosphodiester groups are balanced by cations (“Detection of oligonucleotide duplex forms by ion-spray mass spectrometry”; Ganem B. et al.; Tetrahedron Lett., 34(9), 1445-8, 1993; “Direct observation of a DNA quadruplex by electrospray ionization mass spectrometry,” Goodlett, David R. et al.; Biol. Mass Spectrom., 22(3), 181-3, 1993). The severe problem with ESI of macromolecules is that in general, a multiplicity of charged charge states (q=+e or −e, wherein e is the charge of a single electron) are formed. When the objective is only to determine the mass of a single macromolecule type, the measured specific charges q/m, 2q/m, 3q/m, etc. can usually be deciphered to deduce the sought mass. However when the input sample is a Pop, the combination of hundreds of distinct masses with multiple charges states is not decipherable.

Time of flight (TOF) systems are most popular in trials with DNAs, because of the low cost and mechanical simplicity relative to other MS methods, especially the FT-ICR MS with their expensive magnets. Analytes are ionized with high simultaneity, electrostatically accelerated, acquire spatial separations reflecting their velocity differences in a long drift tube, and the time to impact of analyses is measured. The q/m can then be calculated for the calibrated instrument (“Matrix-assisted laser desorption/ionization mass spectrometry of biopolymers,” Hillenkamp, Franz et at; Annal. Chem., 63(24), 1193A-1203A. 1991). A precise start is required for high temporal resolution detection of fractionation output. TOF MS strategies for DNA are built on successes with proteins, with injection/ionization implemented by either electrospray or mass ablation laser desorption ionization (MALDI).

For MALDI, macromolecules embedded in a matrix of low mass molecules are ejected into the vacuum in a plume of vaporized matrix. A problem encountered with MALDI of simplex DNA is breakage. Initial trials with short homogenous simplexes revealed severe fragmentation problems (“Matrix-assisted laser-desorption mass spectrometry of DNA using an infrared free-electron laser,” Haugland, R. F. et al.; Proc. SPIE-Int. Soc. Opt. Eng., 1854 (FEL), 1993). Two distinct molecules of lower mass are split off by a break in the deoxyribose-phosphodiester backbone of single stranded DNA. Even for a homogenous population of single stranded DNAs, the resultant fragments have a broad range of lower masses. For projected heterogeneous single stranded Pop as inputs for sequencing, lower mass members will be within the fragmentation background and thus harder to recognize. Considerable current research is consequently devoted to searches for alternative matrices and conditions minimizing fragmentation (“Matrix-assisted laser desorption ionization of oligonucleotides with various matrixes,” Tang, K et al.; Rapid Commun. Mass Spectrom., 7(10), 943-8, 1993; “Laser ablation of intact massive biomolecules,” Williams, P. et al., Laser ablation, Mechanisms and applications, Proceedings of Conference: Workshop on Laser Ablation: Mechanism and Applications, Oak Ridge, Tenn. (USA), Apr. 8-10, 1991, J. Am. Chem. Soc., 115(2), 803-4, 1993; “Matrix-assisted laser desorption time-of-flight mass spectrometry of oligonucleotides using 3-hydroxypicolinic acid as an ultraviolet-sensitive matrix,” Wu, Kuang Jen et al., Rapid Commun. Mass Spectrom., 7(2), 142-6, 1993).

A critical problem with MALDI is that the efficiency of injection/ionization is in the range of 10⁻⁴ per macromolecule. This very low efficiency in part reflects trade offs between better ionization and decreasing fragmentation. It limits output signals and forces multiple TOF shots to acquire a useful averaged output. To increase ionization there is an exploration of the use of adducts to DNA, which can be efficiently ionized with minimal concurrent macromolecular fragmentation. The prior art labels considered for MS implementations are ionized by ultraviolet or less energetic photons with ionization resulting from multi-photon excitation and ejection of an electron. (“A novel vacuum ultraviolet ionizer mass spectrometer for DNA sequencing,”; Chen, C. H. et al., Int. J. Genome Res. 1(1), 2543, 1992; “Laser mass spectrometry for biopolymers,” Tang, K. et al., Int. Phys. Conf. Ser. 128 (Resonance Ionization Spectroscopy 1992), pp. 289-92).

A third problem with MALDI is a relatively low velocity band and large velocity dispersion of the ionized DNAs. MALDI is essentially a laser driven chemical explosion. It should be remembered that for DNA fragments consisting of say 500 bases, the mass is very large, say m=150,000. When accelerated by a 50 kV potential typical for current TOF-MS devices, the final velocity is relatively low, v/c=2*10⁻⁵. Thus, the ions are very slow, v=5 km/sec, i.e. comparable with velocity of ions from laser driven chemical explosion.

There are several other causes of mass band broadening affecting even homogeneous macromolecule populations. There may be small mass decreases resulting from ionization chemistries which do not however break the macromolecule apart. There is the presence of 1% C¹³ among the prevalent C¹², with their ratio having statistical variation in the population. There is the statistical variation in counter ion binding at charged sites. For nucleic acids, each phosphodiester group can bind two protons (H+) or other cations. In general, the half width of the isotopic and cation broadening effects will diminish for longer DNAs, following the decrease in N^{-½} as the number N of involved sites increases.

Some partitioning of the electrostatic accelerating energy between linear and angular momentum modes can be anticipated. When charge is not symmetrically distributed with respect to the center of mass of a macromolecule, there is an applied torque during linear acceleration leading to angular momentum. Among a heterogeneously oriented population, the angular momentum acquired will vary with orientation of each macromolecule with respect to the accelerating field. Due to combinations of these effects and thermal broadening, TOF resolution of strands differing by a subunit has only been accomplished for short synthetic polymers.

Two processes have been proposed for reduction of the width of fragment bands (“Detection of electrospray ionization using a quadrupole ion trap storage/reflection time-of-flight mass spectrometer,” Michael, Steven M. et al., Anal. Chem. 65, pp. 2614-20, 1993; “Method for the electrospray ionization of highly conductive aqueous solutions,” Chowdhury, Swapan K et al., Anal. Chem., 63(15), 1660-4, 1991). Ion traps can be used to accumulate charged macromolecules, therein cool them through collisions with noble gases, and finally synchronously eject them into the TOF stage. The second process is the use of electrostatic reflector fields during the TOF stage. The faster macromolecules of a single q/m band penetrate more deeply into an electrostatic field before resection, and thus lose some of their temporal lead over their slower cohort.

A final problem area is detector sensitivity and longevity. Ionization detectors have good temporal resolution. However, ionization is most efficient for impacting ions with velocities comparable with those of electrons in the target. This condition is not satisfied by DNA ions accelerated in TOF MS, contributing to low detection efficiencies. This very low detection efficiency compounded with low ionization during injection leads to poor data acquisition for DNAs. A longevity problem with TOF detectors is due to the large masses of analyzed macromolecules. Impacting macromolecules accumulate on the detector surface and severely compromise efficiency as a near confluent film of debris accumulates.

Labels allow for high sensitivity detection; quantitation of target molecules within complex mixtures; and purifications through affinity chromatography. Labels incorporated into nucleic acids and other macromolecules include: biotinyl groups for purification and non-covalent binding of secondary reporters; fluors, stable isotopes and radioisotopes for purposes of detection; chelating adducts holding multivalent anions, lanthanides in particular, to support fluorescence detection strategies; and release tags, supporting a strategy in which small reporter molecules are split off macromolecules for quantitation by gas chromatography and/or MS (Giese, U.S. Pat. No. 4,709,016, “Molecular analytical release tags and their use in chemical analysis”).

Metallic clusters can serve as labels. The 11 gold atom cluster, undecagold, has been used to label both DNAs and proteins for scanning transmission electron microscopy, STEM Hainfield, “Antibody-gold cluster conjugates useful for tumor imaging, diagnosis and therapy and electron microscopy, diagnostic technique or antigen localization study”). The utility of clusters with high Z (atomic charge) is the high contrast they provide. Clusters containing 55 gold atoms and as many as 309 platinum atoms have also been prepared, though not as yet used as labels (“Electronic structure and bonding of the metal cluster compound Au⁵⁵ (PPh₃)¹²C₁₆,” Thiel et al., Z. Phys., D. (May 1993), v. 26(14) pp. 162-165; “Advances in research on clusters of transition metal atoms,” Whetten et al., Surface Science (June 1985), vol.156, pt.1, pp. 8-35).

Systems which can support discrimination of co-resident label distributions are particularly useful. For any fractionation modality, run-to-run system variations are eliminated when co-resident analyte populations can be co-processed to increase accuracy. This capacity is supported by commercial gel electrophoretic DNA sequencing systems, in which the four Pop are labeled with distinguishable fluors, pooled, co-fractionated, and the members of the Pop members recognized by the combination of in-gel mobility and distinguishing fluorescence. Typically, these systems have sensitivities in the picomole range and only a few co-resident labels can be used because of the broad fluorescence bandwidth. Due to low efficiency of the injection process, DNA sequencing using MS benefits from detection methods requires the highest possible sensitivity.

The use of multiple photon emitting isotopes as labels is described in commonly owned U.S. Pat. No. 5,532,122, WO 97/16746, and WO 98/02750, incorporated herein by reference. Positron-gamma (PG) emitting and electron capture (EC) isotopes have many members that are compatible with ultra-sensitive quantitation by Multi Photon Detection (MPD) systems. The MPD systems achieve extraordinary background rejection by accepting only events which have a coincident multi-photon emission signature of the isotopic label utilized. Sensitivities of 10⁻²¹ moles have been achieved for I¹²⁵ with linearity in detection over a million fold range.

### SUMMARY OF THE INVENTION

This invention satisfies a long felt need for methods for improving the identification of macromolecules in mass spectroscopy, by decreasing breakage, providing for attainment of a single charge state, and increasing sensitivity.

This invention permits success where previous efforts at sequencing long strands of DNA have failed, despite extensive experimentation directed toward that goal. The invention is contrary to the teachings of the prior art requiring the use of single stranded DNA for sequencing. The invention solves previously unrecognized problems in mass balancing duplex DNA.

This invention solves problems previously thought to be insoluble, such as mass band broadening due to mass decreases from ionization, isotopic variation, the heterogeneous binding of cations by phosphodiester moieties in the DNA backbone, tumbling of long molecules upon acceleration, inefficient ionization of macromolecules such as DNA, fouling of detector surfaces, and extensive breakage of single stranded DNA. This invention avoids the need for huge magnets as in FT-ICR and eliminates the multiple charge states resulting from its use in conjunction with ESI, without loss of ability.

Use of mass spectroscopy for sequencing DNA presents advantages over polyacrylamide gel electrophoresis in that larger molecules are more easily distinguished. An embodiment of the method entails running a Sanger sequencing polymerase reaction using a single-stranded template of interest, wherein dideoxynucleotides are used to stop synthesis of the complementary strand at each possible position along the template. A population of molecules are generated that differ in length and mass according to how many normal deoxynucleotides were incorporated before the terminator. Mass spectroscopy may be used to distinguish which dideoxynucleotides were incorporated at a specific position because the different species of dideoxynucleotides, i.e. ddATP, ddCTP, ddGTP and ddTTP, are labeled with different isotopes. By comparing which isotope was incorporated into each member of the population of a different mass or length, one can determine the sequence of the original template. Advantageously the detection system employs MultiPhoton Detector (MPD) technology as in U.S. Pat. No. 5,532,122.

Prior art techniques suffer from instability of the DNA fragments in the mass spectrophotometer. According to the invention, MPD technology is sensitive enough to allow use of double-stranded population of DNA molecules resulting from the sequencing reaction, thereby increasing stability compared to single-stranded molecule. Using double stranded DNA doubles molecular masses and reduces sensitivity so is counter-intuitive. Detector longevity is addressed by de-coupling the fractionation and detection steps of the total MS system.

According to the invention, a method of sequencing a nucleic acid of interest comprises:

(a) providing four populations of pluralities of duplex nucleic acids, each nucleic acid having a common end and a terminal base at the other end, and a length corresponding to the position of the terminal base in the nucleic acid of interest, the duplex nucleic acids having an ionization target, and a detection label associated with the termination base,

(b) ionizing the ionizing targets of the populations of duplex nucleic acid with an ionizing agent,

(c) fractionating the populations of duplex nucleic acid using mass spectroscopy,

(d) for each duplex nucleic acid, resolving a single ionization state, identifying the terminal base by means of the detection label, and determining the sequence length based on mass.

The target nucleic acid has a sequence length greater than about 30 bases, preferably greater than about 300 bases, and may be as long as 400 bases or longer than 1000 bases.

The mass spectroscopy includes spatially resolving mass spectrosopy. The ionization label preferably comprises a high Z atom susceptible to ionization by X-rays, such as an undecagold cluster, or a cluster of a platinide, a lanthanide, or a combination. The ionizing agent may be high energy photons from an X-ray tube with cathode of atomic number Z+1 or other element whose K or L shell X-rays have slightly greater energy that the K or L shell edge of the ionization target. Where the ionization target comprises gold the cathode for X-ray emission may be mercury, thallium, strontium, or yttrium. Where the ionization target comprises a platinide, the cathode for X-ray emission may be the platinide with next highest atomic number.

The ionization target may react when excited by photons to produce a charged component connected to the duplex nucleic acid, such as triarylmethyl compounds, o-nitrobenzylcarbamate, m-alkoxybenzylcarbamate, thiocarbamate, or o-nitrobenzyldithiocarbamate.

The method may comprise decoupling detection from fractionation by directing the fractions onto a target plate, moving or removing the plate, and subsequently detecting the fractions on the plate. The method may comprise spinning the target plate.

The detection may be by atomic force, scanning tunneling or near field emission microscopies, or other quantitative imaging. Where the detection label comprises at least one cluster of high Z metal, the detecting may comprise scanning transmission electron microscopy. Where the detection label comprises a fluor, the target plate may be low Z substrate such as LiH, and the detecting may comprise detecting phosphorescence or fluorescence on the substrate.

The detection label preferably comprises a multiple photon emitting radioisotope, and the detecting comprises multiphoton detection. The radioisotope may be an electron capture isotope of Re, Os, Ir, Pt, or Au.

The method may comprise replacing hydrogen ions with lithium cations at the phosphodiester groups of the nucleic acids to reduce mass variation.

The step of providing populations of duplex nucleic acid may comprise: providing a simplex template of the nucleic acid of interest, providing a primer complementary to a portion of the simplex template, extension bases, and termination bases for A, T, G, and C, providing the termination bases with a detection label, providing the duplex nucleic acids with an ionization target, catalyzing extension of the primer with a sequence complementary to the simplex template to form a nucleic acid construct having duplex nucleic acid regions, and digesting the nucleic acid construct with a nuclease to produce four populations of pluralities of duplex nucleic acids having termination bases at the terminal end and lengths corresponding to the positions of the termination bases. The method may further comprise removing impurities by providing the duplex nucleic acid with a ligand, providing a substrate with a receptor, binding the duplex nucleic acid to the substrate, and washing away impurities.

The method may further comprise balancing the mass of the duplex nucleic acids by increasing the mass of the A or T extension bases by one amu by isotopic substitution at a stable position of the base. The isotopic substitution in each A or T may be replacing a single hydrogen atom with deuterium, replacing a single C¹² atom with C¹³, replacing a single N¹⁴ atom with N¹⁵, replacing a single O¹⁶ atom with O¹⁷, or replacing a single P³¹ atom with P³². The method may further comprise providing three sets of populations of duplex nucleic acid, a first set with no mass compensation, a second set with mass compensated by 1 amu, and a third set with mass over-compensated by 2 amu substitution, and obtaining redundant information about the mass of the fragments. The first set may have non-substituted hydrogen, carbon, oxygen, or phosphorous, the second set a single deuterium, C¹³, O¹⁷, or P³² substitution, and the third set a single tritium, C¹⁴, O¹⁸, or P³³ substitution, respectively.

More broadly, the invention relates to a method of determining the mass of a macromolecule comprising:

(a) providing the macromolecule with an ionization target and a detection label,

(b) ionizing the ionizing targets with an ionizing agent to provide a single ionization state,

(c) subjecting the macromolecule to fractionation by mass spectroscopy, and

(d) detecting the detection label and determining the mass of the macromolecule.

The ionization target, ionizing agent, detection label, fractionation, and detection may all be as described for the specific embodiment of DNA sequencing.

The invention also encompasses a device for sequencing DNA comprising:

(a) means for providing four populations of pluralities of duplex nucleic acids, each nucleic acid having a common end and a terminal base at the other end, and a length corresponding to the position of the terminal base in the nucleic acid of interest, the duplex nucleic acids having an ionization target, and a detection label associated with the termination base,

(b) means for ionizing the ionizing targets of the populations of duplex nucleic acid with an ionizing agent,

(c) means for fractionating the populations of duplex nucleic acid using mass spectroscopy,

(d) means for identifying the terminal base of each duplex nucleic acid, by means of the detection label, and determining the sequence length based on mass.

Another aspect of the invention is a population of duplex DNA molecules of lengths greater than about 50 bases, or preferably a length greater than about 50 bases corresponding to the sequence of a nucleic acid of interest, each molecule having a common end and a terminal base at the other end, and a length corresponding to the position of the terminal base in the nucleic acid of interest, and each molecule having an ionization target and a detection label associated with the terminal base, each molecule being susceptible to ionization to produce essentially a single charge state for that length. Preferably the molecules of the population are mass balanced by isotopic substitution so that the mass of the A−T pairs equals that of the G−C pairs.

Further objectives and advantages will become apparent from a consideration of the description.

### DETAILED DESCRIPTION OF THE PREFERRED EMBODIMENTS

In describing preferred embodiments of the present invention illustrated in the drawings, specific terminology is employed for the sake of clarity. However, the invention is not intended to be limited to the specific terminology so selected, and it is to be understood that each specific element includes all technical equivalents which operate in a similar manner to accomplish a similar purpose.

Two advantages of Pop fractionation by MS are the high speed (milliseconds as contrasted to minutes for gel electrophoresis), and the potential for much longer sequence reads. In MS of low molecular mass analyses (m<1000 amu), resolutions of Δm/m=0.0001 are commonly achievable, where m and m+▴m are the masses of two analyses differing in mass by Δm. With comparable resolution for Pop analyses, this translates into sequence reads of a few thousand bases. Longer reads bring significant economies to large sequencing projects by reducing the number of Pop which must be prepared to cover the subject chromosome and support assembly of its entire sequence.

Unfortunately, existing methods for MS of high mass DNA molecules show that it is increasingly difficult to have good mass resolution for m>100,000 amu, i.e. about 300 base units due to problems in analyses and instrumentation. Depending on the MS instrument utilized, the problems include:

low efficiency of injection into the vacuum and ionization;

occurrence of multiple ionization states;

breakage of macromolecules during injection and ionization;

low mass resolution in Time Of Flight (TOF) MS instruments;

lack of appropriate detectors for slow moving macromolecules.

This invention relates to improvements in the mass spectroscopy (MS) of macromolecules, with sequencing of DNA being a motivating application. In a first embodiment, the utilities of Pop comprised of DNA duplexes as contrasted to single stranded DNAs include a greatly reduced susceptibility to macromolecular breakage during energetic processes. Duplex DNA does not split apart despite a single stranded break, because the complementary intact strand maintains the continuity of the two duplex segments. More generally, a duplex DNA can suffer numerous single strand breaks but will only be split when a pair of breaks is on opposite strands and within a few subunits of one another. The substitution of duplex DNAs for single stranded DNAs in MS determinations facilitates sequencing by preserving the mass of members of the input Pop. This substitution is particularly beneficial to MALDI implementations and more energetic ionization processes.

Thus, advantages follow from substituting DNA duplexes for the single strands heretofore utilized in Maxam-Gilbert or Sanger sequencing strategies, with a resultant expansion in the DNA adducts suitable as targets for selective and efficient ionization. The advantages include decreased analyte breakage and more reliable mass band discrimination when using duplex DNA.

Pops comprised of either single stranded (simplex) or duplex DNA can be generated by several different techniques known to those skilled in nucleic acid methodologies. Sanger methods are preferred. The Sanger Pop production begins with the binding by base pairing of a short single stranded DNA, the initial reaction prime, at a chosen site on the single stranded DNA template to be sequenced. The additions of new subunits at the 3′ hydroxyl end of primers and new 3′ ends thus generated are catalyzed by a DNA polymerase. The choice of subunit is strongly determined by the template, manifesting in the restriction to A base paired only with T and G base paired only with C during the polymerization. Radioisotopic or other, e.g. fluorescent labels may be incorporated into primers, the added subunits or “terminator” bases for subsequent purposes of product purification or detection, as further detailed below. The most commonly used terminators are 2′,3′-dideoxyribose analogues of the normal 2′ deoxyribose precursors. The ratio of a normal subunit and its dideoxyribose analogue are chosen to achieve the complete distribution of DNA fragments with the length up to about 400-1000 bases of template.

Prior to fractionation, reaction debris may be eliminated by a variety of procedures. One family of procedures has in common the use of the high specificity and affinity of the protein streptavidin for biotinyl groups. The streptavidin is fixed to an appropriate matrix or support. The biotin is covalently linked to either the templates or their bound complement strand, with the linking chemistry performed prior to Pop production biochemistry. The DNA is captured to the solid phase streptavidin, and reaction debris are eliminated through a series of washes. The purified DNAs are then released into an appropriate solution, and the four Pop fractionations implemented.

Limits on gel electrophoretic fractionation of DNA arise because the spacing between successive bands does not decrease uniformly, reflecting intramolecular subunit interactions altering the compactness of strands and hence their mobility. This effect is absent for MS fractionations of single stranded Pop, as intramolecular interactions do not decrease mass. There remains however, the unpredictability due to the differing subunit masses (see Table 1). More specifically, when simplex DNA is used the uncertainty in the mass increment of successive larger fragments can be as much as m[C]−m[G]=30 amu. This leads to mass uncertainty of 0.5*(m[C]−m[G])/(m[C]+m[G])=2.5% of the incremental mass. An advantage of MS in DNA sequencing is a mass resolution of 0.01%. Reduction in uncertainty due to subunit mass differences is a fundamental benefit of the disclosures below.

A second aspect of the invention is the more reliable fragment band discrimination when Pop are used. In the combined analysis of data from the four Pop, the critical question at each subunit read step is: which one of the fractionated Pop contains a band corresponding to a mass one subunit longer than the band previously read? The incremental mass uncertainty is as much as m[G]−m[C]=329.2−289.2=30 amu, or about 10% of the mass of a subunit addition to a single stranded DNA. This type of uncertainty is a fundamental limiting factor on the use of MS in single stranded DNA analysis. Reducing uncertainty in the masses of successive fragments thus increases sequence read lengths and efficient chromosome sequencing.

Replacement of a single stranded DNA Pop with a duplex DNA provides a significant reduction in the incremental mass uncertainty. The lowest mass member in a Pop is the duplex form of the primer extended only by a terminator subunit. For all longer members within the four Pop compared, the incremental mass due to the addition of a C+G pair is 618.4 amu and for the A+T pair 617.4 (Table 2). The incremental uncertainty thus corresponds to only one amu or about 0.16% of the mass of a base pair added, as contrasted with 10% for the subunit addition to DNA simplexes. The improved mass resolution provided by using Pop of DNA duplexes as contrasted to simplexes is realized as the problem of bandwidths is overcome by cooling in ion traps, electrostatic reflector fields and other means.

TABLE 2
Masses of the subunit pairs (amu)

mass [G + C] = 329.2 + 289.2 = 618.4
mass [A + T] = 313.2 + 304.2 = 617.4

The one amu difference between A−T and G−C subunit pairs can be substantially eliminated by mass balancing. In one approach, the precursors of the A and T subunits for the polymerase reaction have a single isotopic substitution that adds one amu—for example, deuterium for hydrogen (but only at a position non-ionizable in aqueous solution), carbon-13 for carbon-12, nitrogen-15 for nitrogen-14 or phosphorus-32 for phosphorus-31. More generally, there is an array of isotopes available to achieve not only mass balancing of A+T and G+C pairs, but also mass balancing when a useful subunit analogue maybe substituted for the normal one. Chemical steps for preparing such isotopically modified DNA precursors are known in the art.

In a second approach, the template strand can be prepared with the A and T subunits having one of the isotopic substitutions. This preparation can be achieved by performing the polymerase chain reaction on the DNA segment to serve as template, and incorporating the isotopically heavier A and T subunit precursors. The product population of identical duplexes thus generated will contain the isotopically heavier A and T in both strands. Subsequently the Sanger biochemistry can be performed with ordinary A and T subunit precursors. The production and use of the necessary isotopically substituted precursors would be warranted, however, only if the one amu positional certainty becomes in practice more deleterious than the other band broadening factors described above.

This type of base pair mass balancing confers another surprising advantage. The modification of nucleic acids by the adduction of small chemical groups to them is one of the mechanisms through which gene expression is regulated. The methylation of C subunits which adds 15 amu is one of the more common modifications. According to the invention, the presence of a methylated subunit in the template would shift the masses of the corresponding band and all subsequent bands by 15 amu. The regulating methylation site would thus be unambiguously displayed as opposed to MS of Pop comprised of simplexes.

Another innovative isotopic substitution strategy includes using phosphorus P³¹, P³² and P³³ sequentially in production of duplex Pop. This leads to three sequence reads wherein the one unit mass difference is uncompensated, compensated and over-compensated, respectively. Comparison of these three reads provides both increased redundancy and the possibility of calculating and correcting mass broadening due to other factors.

Another advantage of the invention is the expansion of ionization modes which can be considered, when single strand breakage will not culminate in macromolecule breakage. The use of DNA adducts carrying high atomic number (Z) atoms or their clusters then becomes reasonable, to achieve selective ionization by single X-ray photons. High Z in this context means greater than about 140 amu, preferably greater than 180 amu. For lower Z atoms, the prevalent energy absorption mechanism is through the non-ionizing Compton effect: an electron is excited to a higher energy orbital and a photon with reduced energy is emitted. For higher Z atoms, X-rays are absorbed through the photoelectric effect, i.e. ejection of electrons predominantly from K and L shells. Thus, a relevant physical quantity is the photoelectric effect cross-section. Empirically, it is proportional to Z^{3.4}, i.e. probability of ionizing a molecule is the sum of the Z^{3.4} for all its atoms. It is this strong dependence on Z that motivates consideration of high Z targets as labels for macromolecules. The use of X-rays to induce ionization is not rational in the case of simplex DNA fragments, because of the inherent higher strand breakage probability. In contrast, X-ray usage is rational for duplex DNAs (and also for proteins with their multiple intramolecular structure-conserving interactions). While some bond breaks may result from the chemical reactivity of Compton effect excitations, macromolecule spitting is probable only at much higher X-ray exposures than necessary for MS ionization.

The quantitative advantage is illustrated for the case of undecagold. The mass of undecagold is 11×197=2167 amu, less than that of four base pairs with m=618. Thus it is not a deleteriously large contribution to the total mass of a long DNA duplex. The Z^{3.4} for undecagold and a single DNA base pair are about 32.5 million and 51,000 respectively, a factor of more than 600. Thus for DNAs less than 600 base pairs long carrying one undecagold label, the labels have a higher probability of ionization than the host DNA duplexes with exposures only sufficient to ionize a small-fraction of the Pop, say 10%. The concurrent probability of double ionization is very low, about 1%. Thus undecagold is a preferred label for achieving a preponderant q=1+ charge state among the DNAs ionized. Multiple undecagold labels as desired can be incorporated into primers or terminators for the sequencing biochemistry, to extend its utility into the few kilobase range. Alternatively, a single more massive high Z cluster can be utilized. The immediate practical advantage of undecagold is its commercial availability with a linker supporting covalent attachment to macromolecules. Other heavy metal labels, including lanthanides and other platinides (Re, Os, Ir) may be used.

Preferred ionization strategies for high Z labels are now disclosed. Atoms have well deemed edges in their X-ray absorption spectrum at which absorption is locally maximal. Optimal ionization of a target is achieved for X-rays with energies just higher than the edge. The most compact X-ray source is a conventional X-ray tube. An emitting cathode made of material Z+1 with respect to the Z target generally provides X-rays with desired energy. For a gold target, this calls for a mercury cathode. The use of a mercury cathode with an appropriate cooling system may be adequate in spite of Hg low melting temperature. However, cryogenic cooling, e.g. with liquid nitrogen, is somewhat onerous. Thus, other alternatives should also be considered. The next higher Z atom is thallium (Tl), with a suitable melting point of 303° C. However thallium is highly toxic and another alternative should thus be considered. For gold the L shell edge is about 80 keV and the K shell energy edge is about 14.37 keV. With a gold K-shell electron as ionization target, the use of strontium (E₁=16.01 keV) or Yttrium (E₁=17.05 keV) as cathodes would be suitable. These metals have suitably high melting points of 769 and 293° C., respectively.

Metal clusters of lanthanide and platinide atoms have been prepared and could be attached to macromolecules, with the advantage that there is a large choice of suitable metallic cathodes for use as X-ray sources for label ionization. If an element with atomic number Z is the macromolecule's label, the cathode should be made of the element with atomic number Z+1. Fortunately, all platinides and lanthanides have very high melting temperature and there are many suitable Z label/Z+1 cathode pairs.

For very high throughput operation, pulsed X-ray sources at synchrotron radiation facilities could be useful. The gold K shell absorption edge is low enough so that a reasonably high flux of synchrotron radiation photons is available, about 109 photons/burst/cm² and monochromatization by Bragg reflection is possible with efficiency of a few percent. Burst duration is very short, a fraction of a microsecond. It thus can provide the start trigger for TOF MS analysis. An appropriately filtered beam of brehmstrahlung photons can be used, produced by a pulsed electron beam passing though a beryllium foil.

In another type of ionization target, selective ionization is induced by light in the visible to ultraviolet range. For example, some molecules have a general structural feature of a central carbon atom with three strong bonds to three energy “antennas” and a fourth much weaker bond to an atom or group(G). The G group is split off with concomitant ionization when photon energy is absorbed in the antennas. The antennas are typically in the substituted benzyl family, with a variety of substitutions for hydrogens controlling solubilities and absorption spectra. Splitting times of the order of 10⁻⁹ have been demonstrated, in demonstrations of pH changes induced by separation of the G group, a hydroxyl ion (“Light induced reversible pH changes,” Irie, J. Am. Chem. Soc. 105, 2078-2079, 1983; “Photogenerated amines and their use in the design of a positive-tone resist material based on electrophilic aromatic substitution,” Matuszczak et al, J. Mater. Chem., 1(6), 1045-50, 1991). Using this approach, a laser pulse can serve as the timing trigger for TOF MS. For application to macromolecules the G group is the linker to the macromolecule, including DNAs. Specifically, 4,4′-bis(diphenylamino)triphenylmethane-G is promising where G is an ester linkage to the macromolecule.

For DNA applications, the ionization target complex is most simply attached to the prier for the Sanger reactions. This strategy differs significantly from approaches in which ionization is sought by ejection of the very light electron. Charge recombination/neutralization powered by electrostatic attraction of atomic mass or heavier charged groups is much slower than those involving capture of the electrons. Hence more macromolecules will retain their charge during the electrostatic acceleration phase of MS Such charge retention can have a significant impact on the amount of primary sample which must be prepared for MS analysis.

Another class of ionization targets has the common feature of a potential for a highly exothermic scission when stimulated, which drives the production of charged products, Such reactivity would itself be considered a highly negative feature, compromising the integrity of single stranded DNAs. The utility of this exothermic character is thus non-obvious, until the robustness of duplex DNA for MS is first recognized. Promising groups for this family of labels are the o-nitrobenylcarbamates. They are easy to synthesize as adducts to primers. The reaction can be stimulated with ultraviolet photons and proceeds with formation of carbon dioxide as one of the final products. This reaction has been used to trigger the fast formation of a base for purposes of microlithography. A prevalent retention of a negative charge by the nitrosobenzaldehyde and a positive charge by the group remaining attached to the DNA is expected. The intramolecular reaction is exothermic and stimulated with a quantum efficiency of 0.65 by ultraviolet light photons. Similar useful properties are expected for exothermic reactions of m-alkoxybenylcarbamates, thiocarbamates and o-nitrobenzyldithiocarbamates.

The- detection labels remaining after MS fractionations may be used with spatially resolving MS (SR-MS)instruments. Historically, SR-MS were the first MS implemented, using a magnetic field transverse to the particle trajectory to bend trajectories of analyses with different q/m to detection positions. However, they had been used only to fractionate low mass molecules which gave good impact signals. SR-MS is disfavored for fractionation of macromolecules, because of perceived technical and cost advantages of TOF-MS Thus, the of SR-MS is counter-intuitive and suitable only with concurrent use of the innovations disclosed herein, i.e. a use of labels allowing decoupling of the MS fractionation and detection stages.

In the following implementations, the Pop members are targeted by an SR MS at a movable and/or removable plate. The plate preferably has no detection capability by itself. Rather it is used to transfer the deposited Pop members to secondary detection systems. Such de-coupling has multiple benefits:

1) it avoids the diminishing efficiency expected for higher mass macromolecules with conventional TOF MS detectors;

2) the detector itself does not become crusted with analyte debris;

3) co-resident Pop patterns can be analyzed, when the inputs have distinguishing labels;

4) the input sample need not be pure, so long as the detection label distinguishes its macromolecule from contaminants; and

5) a variety of detectors can be used for plate readout, dependent upon the properties of the macromolecules and their labels.

In some implementations, a rigid plate body supports a thin removable plastic layer on which the analytes are deposited. Plastics are desirable for their low atomic number atoms, which provide minimal absorption of radioisotopic emissions and/or minimal scattering of electrons in contrast to high Z labels. Generally, only the most sensitive, i.e. very low background, spatially resolving detection systems are desirable for readout of the plated analyses. The detection system should be compatible with the plurality of labels carried by the macromolecules and should allow high dynamic range.

For some macromolecules no label is necessary, for example, if quantitative imaging can be accomplished by techniques of atomic force, scanning tunneling or near field emission microscopies. For this detector implementation however, great care has to be taken to provide atomically flat surfaces.

Furthermore, as the plate is already in a vacuum and measurement can be stationary, electron induced phosphorescence or fluoresence of appropriate macromolecules and can also be easily implemented. However, excitations by electrons even if they lead to higher flux of re-emitted photons is much less specific, e.g. the problem of fluorescence of the substrate material may be overwhelming. This can be minimized by using the lowest atomic number solid substrate available, such as lithium hydride, LiH.

For macromolecules with high Z labels including metal clusters, an appropriate readout instrumentation is the scanning transmission electron microscope, STEM. Its electron energies can be adjusted to scatter preferentially from atoms of a chosen Z, and quantitations are much less dependent on plate surface imperfections than the aforementioned scanning modalities. In particular, the STEM has been used effectively for macromolecules labeled with undecagold. For undecagold additional silver deposition can be effected, so that even the visible light microscope suffices as a readout instrument.

A general advantage of the scanning microscopy systems is that sub-micron spatial resolution is easily achieved. Thus the bands from fractionated Pop can be deposited on a much smaller surface area than that needed for prior art SR-MS instruments, in which individual detector element dimensions are orders of magnitude larger than the spatial resolution of current scanning microscopes. There is a tradeoff between sensitivity and STEM scan speed. Thus, specific techniques permitting the fast read-out or concurrent read-out at many microscope stations are preferred. According to the invention, for example, at least pico-moles of MS fractionation output are available (for lowest abundance fragments), leading to rather large, about tens of micrograms input of DNA material per shot.

Even greater benefits may be achieved through the use of macromolecules labeled with EC and/or PG isotopes. Decays of PG emitting isotopes manifest in the appearance of a nuclear gamma, a positron ionization track, and two opposed 511 keV photons as the positron and an electron annihilate. Among the EC isotopes, the majority have coincident emission of X-ray and gamma photons. Among the EC and PG isotopes together, choices of label half-life can be made to best match the desired throughput of the total MS sequencing system. The MPD system supports the simultaneous quantitation of multiple isotopes which can be distinguished by the energies of their monochromatic nuclear gamma lines. Relevant PG and EC radioisotopes include gold and platinides isotopes and over 20 isotopes of lanthanides.

In the case of duplex DNA analysis by MS, multiple Pop can be deposited on a single plate, with subsequent simultaneous readout by a MPD system distinguishing isotopes by the energy of their nuclear gammas. For example, Table 3 discloses appropriate isotopes for the platinides family and gold. Only EC isotopes with reasonable lifetime longer than a few hours are quoted. For each of the elements with the exception of osmium, there are a sufficient number of isotopes to label each of the four Pop in a sequencing with different, easily distinguishable-isotopes.

TABLE 3
The EC isotopes (half-lives in parentheses)

| | |
|---|---|
| Renium | Re¹⁸¹ (20 h), Re^{182 m} (13 h), Re^{184 m} (2.2 d), Re¹⁸⁴ (50 d), Re¹⁸⁶ |
| | (90 h) |
| Osmi- | Os^{183 m} (10 h), Os¹⁸³ (12 h), Os¹⁸⁵ (94 d) |
| um | |
| Iridium | Ir¹⁸⁵ (15 h), Ir¹⁸⁶ (5 h), Ir¹⁸⁷ (12 h), Ir¹⁸⁸ (41 h), Ir¹⁸⁹ (11 d), ^{190 m} |
| | (3.2 h), Ir¹⁹² (74 d) |
| Plati- | Pt¹⁸⁶ (25 h), Pt¹⁸⁸ (10 d), Pt¹⁸⁹ (11 h), Pt¹⁹¹ (3 d), Pt¹⁹³ |
| num | (<550 y) |
| Gold | Au¹⁹¹ (3.2 h), Au¹⁹² (4.8 h), Au¹⁹³ (15.8 h), Au¹⁹⁴ (39 h), Au¹⁹⁵ |
| | (200 d) Au¹⁹⁶ (5.55 d) |

It is evident that the disclosures above can be implemented in a variety of combinations towards the goal of sequencing DNA with fractionation by MS, or measuring the mass of other macromolecules. A limiting feature in current technologies is the efficient attainment of a single charged state among members of a duplex Pop. The FT-ICR MS provides the highest sensitivity and q/m resolution.

The M13 DNA template system which is extensively used in sequencing is utilized. The M13 is a single stranded DNA virus with a protein coat which is excreted from the intact bacterial host. The DNA is purified as a template for Sanger biochemistry. As a template, the M13 features a primer binding site adjacent to the DNA segment to be sequenced. A segment of M13 of known sequence is first analyzed. The primer is equipped with both biotin and undecagold labels. The Sanger biochemistry is performed. High atomic number labels, e.g an undecagold label on the primer, are used as an ionization target.

The Sanger reaction products are reacted to produce Pop of duplexes, rather than separating template and product strands as in prior art. The Sanger products comprised of template strands partially converted to duplexes are treated with nuclease S1, which selectively degrades single stranded DNAs. The enzyme's action on the Pop generates populations of duplex Pop and diverse debris. The duplexes are bound to solid phase streptavidin through their biotinyl group and washed free of debris and proteins. Production of the duplexes could be achieved by several different approaches apparent to those of ordinary skill.

Lithium may be used as cation instead of other, more massive cations. Lithium cations are simply provided for example in a penultimate wash with a buffer pH=7 in 0.01 mole lithium acetate. Lithium has the smallest mass of possible monovalent cations for DNA, differing from the hydrogen ion by only two amu. This minimizes mass band broadening due to statistical variations in cation binding among macromolecules of the same mass. The final wash is with a buffer containing 0.001 molar biotin and 0.001 molar lithium acetate, to be performed at pH=7. Competition by the biotin for the streptavidin binding sites releases the biotinylated duplexes from the solid phase.

Undecagold is the preferred selective ionization target. Ionization is achieved by 16 keV X-rays emitted from the strontium cathode of an X-ray tube, with photo-electric effect ejection of an electron being the dominant ionization mode. For q/m readout, the use of a FT-ICR equipped for electrospray ionization (ESI) is disclosed, with the following modifications. The X-ray tube is mounted adjacent to the solution inlet capillary, to serve as an alternative to the electrospray ionization. With an ESI condition chosen as a reference, the effects of increasing X-ray exposure may be calibrated and optimized.

According to the invention, at an X-ray exposure just sufficient to detect ionized DNAs, there is near uniform representation of the Pop members with a q=1+charge state. As exposure is increased, overall signal strength will be increased. A high exposure “regime” is reached at which the DNA multiple charged states start to manifest. This preferentially affects the longer duplexes first, revealed as an ICR signal shifts from q/m analyses to 2q/m, and progressively affecting lower masses as the X-ray flux is increased.

Because the duplexes are not broken at deleterious rates below the high flux regime, there is an absence of a troublesome diffuse background. The control for the benefits of using duplexes is a comparison set of experiments with single stranded Pop, be prepared as described above, except that separation of template and products in alkali solution replaces nuclease S1 treatment.

In order to distinguish between uncharged fractions, the major fraction with a single charge state +1 and the minor fractions with +2, +3, or higher charge states, a calibration procedure is desirable. This method relies on the fact that uncharged molecules are not accelerated, and that the minor fractions have much lower activity, and higher speed. Accordingly, a distribution pattern of the triple, then the double charged molecules reaches the target more quickly than the similar distribution pattern of the single charged molecules. However, as the concentration of the multiply charged molecules is much less than that of the singly charged molecules (e.g. if the likelihood of a single charge is less than about 10%, the likelihood of a double charge is about 1%, or an order of magnitude lower, and in the preferred embodiment where the likelihood of a single charge is only about 1%, the likelihood of a double charge is two orders of magnitude lower). Because the amplitude for-the multiple charged molecules is so much lower than the singly charged molecules, the faster, but lower amplitude pattern of the multiple charged molecules can be matched to the pattern for the singly charged molecules and eliminated from further consideration. This process is repeated for short single charged sequences which may overlap with longer double charged sequences, and very short single charged sequences, which may overlap with triple charged sequences.

The references cited here are incorporated by reference in their entirety as if each were individually incorporated by reference. The embodiments illustrated and discussed in this specification are intended only to teach those skilled in the art the best way known to the inventors to make and use the invention. Nothing in this specification should be considered as limiting the scope of the present invention. Modifications and variations of the above-described embodiments of the invention are possible without departing from the invention, as appreciated by those skilled in the art in light of the above teachings. It is therefore to be understood that, within the scope of the claims and their equivalents, the invention may be practiced otherwise than as specifically described.

What is claimed is:

1. A method of sequencing a nucleic acid of interest comprising:
providing four populations of pluralities of duplex nucleic acids, each nucleic acid having a common end and a terminal base at the other end, and a length corresponding to the position of the terminal base in the nucleic acid of interest, the duplex nucleic acids having an ionization target, and a detection label associated with the termination base,
ionizing the ionizing targets of the populations of duplex nucleic acid with an ionizing agent,
fractionating the populations of duplex nucleic acid using mass spectroscopy,
for each duplex nucleic acid, resolving a single ionization state, identifying the terminal base by means of the detection label, and determining the sequence length based on mass.

2. The method of claim 1, wherein the target nucleic acid has a sequence length greater than about 300 bases.

3. The method of claim 1, wherein the mass spectroscopy is spatially resolving mass spectroscopy.

4. The method of claim 1, wherein the ionization label comprises a high Z atom susceptible to ionization by X-rays.

5. The method of claim 4, wherein the ionization label comprises an undecagold cluster.

6. The method of claim 4, wherein the ionization label is at least one cluster of a platinide, a lanthanide, or a combination.

7. The method of claim 3, wherein the ionizing agent is high energy photons from an X-ray tube with cathode of atomic number Z+1 or other element whose K or L shell X-rays have slightly greater energy that the K or L shell edge of the ionization target.

8. The method of claim 1, wherein the ionization target comprises gold and the cathode for X-ray emission is selected from the group consisting of mercury, thallium, strontium, and yttrium.

9. The method of claim 1, wherein the ionization target comprises a platinide and the cathode for X-ray emission is the platinide with next highest atomic number.

10. The method of claim 1, wherein the ionization target reacts when excited by photons to produce a charged component connected to the duplex nucleic acid.

11. The method of claim 1, wherein the ionization target is selected from the group consisting of triarylmethyl compounds, o-nitrobenzylcarbamate, m-alkoxybenzylcarbamate, thiocarbamate, and o-nitrobenzyldithiocarbamate.

12. The method of claim 1, comprising decoupling detection from fractionation by directing the fractions onto a target plate, moving or removing the plate, and subsequently detecting the fractions on the plate.

13. The method of claim 12, comprising spinning the target plate.

14. The method of claim 1, wherein detection is by atomic force, scanning tunneling or near field emission microscopies, or other quantitative imaging.

15. The method of claim 1, wherein the detection label comprises at least one cluster of high Z metal, and the detecting comprises scanning transmission electron microscopy.

16. The method of claim 1, wherein the detection label comprises a fluor, the target plate is a low Z substrate, and the detecting comprises detecting phosphorescence or fluorescence on the substrate.

17. The method of claim 1, wherein the detection label comprises a multiple photon emitting radioisotope, and the detecting comprises multiphoton detection.

18. The method of claim 17, wherein the radioisotope is an electron capture isotope of Re, Os, Ir, Pt, or Au.

19. The method of claim 1, further comprising replacing hydrogen ions with lithium cations at the phosphodiester groups of the nucleic acids to reduce mass variation.

20. The method of claim 1, wherein the step of providing populations of duplex nucleic acid comprises:
providing a simplex template of the nucleic acid of interest,
providing a primer complementary to a portion of the simplex template, extension bases, and termination bases for A, T, G, and C,
providing the termination bases with a detection label,
providing the duplex nucleic acids with an ionization target,
catalyzing extension of the primer with a sequence complementary to the simplex template to form a nucleic acid construct having duplex nucleic acid regions,
digesting the nucleic acid construct with a nuclease to produce four populations of pluralities of duplex nucleic acids having termination bases at the terminal end and lengths corresponding to the positions of the termination bases,

21. The method of claim 20, further comprising removing impurities by providing the duplex nucleic acid with a ligand, providing a substrate with a receptor, binding the duplex nucleic acid to the substrate, and washing away impurities

22. The method of claim 1, further comprising balancing the mass of the duplex nucleic acids by increasing the mass of the A or T extension bases by one amu by isotopic substitution at a stable position of the base.

23. The method of claim 22, wherein the isotopic substitution in each A or T is selected from the group consisting of replacing a single hydrogen atom with deuterium, replacing a single C¹² atom with C¹³, replacing a single N¹⁴ atom with N¹⁵, replacing a single O¹⁶ atom with O¹⁷, and replacing a single P³¹ atom with P³².

24. The method of claim 22, further comprising providing three sets of populations of duplex nucleic acid, a first set with no mass compensation, a second set with mass compensated by 1 amu, and a third set with mass over-compensated by 2 amu substitution, and obtaining redundant information about the mass of the fragments.

25. The method of claim 24, wherein the first set has non-substituted hydrogen, carbon, oxygen, or phosphorous, the second set has a single deuterium, C¹³, O¹⁷, or P³² substitution, and the third set has a single tritium, C¹⁴, O¹⁸, or P³³ substitution, respectively.

26. A method of determining the mass of a macromolecule comprising:
providing the macromolecule with an ionization target and a detection label,
ionizing the ionizing targets with an ionizing agent to provide essentially a single ionization state,
subjecting the macromolecule to fractionation by mass spectroscopy,
detecting the detection label and determining the mass of the macromolecule.

27. The method of claim 26, wherein the ionization label comprises a high Z atom susceptible to ionization by X-rays.

28. The method of claim 27, wherein the ionization label comprises a cluster of gold, a platinide, a lanthanide, or a combination.

29. The method of claim 27, wherein the ionizing agent is high energy photons from an X-ray tube with cathode of atomic number Z+1 or other element whose K or L shell X-rays have slightly greater energy that the K or L shell edge of the ionization target.

30. The method of claim 29, wherein the ionization target comprises gold and the cathode for X-ray emission is selected from the group consisting of mercury, thallium, strontium, and yttrium.

31. The method of claim 26, wherein the ionization target reacts when excited by photons to produce a charged component connected to the duplex nucleic acid, and is selected from the group consisting of triarylmethyl compounds, o-nitrobenzylcarbamate, m-alkoxybenzylcarbamate, thiocarbamate, and o-nitrobenzyldithiocarbamate.

32. The method of claim 26, comprising decoupling detection front fractionation by directing the fractions onto a target plate, moving or removing the plate, and subsequently detecting the fractions on the plate.

33. The method of claim 32, wherein detection is by atomic force, scanning tunneling or near field emission microscopies, or other quantitative imaging.

34. The method of claim 32, wherein the detection label comprises at least one cluster of high Z metal, and the detecting comprises scanning transmission electron microscopy.

35. The method of claim 26, wherein the detection label comprises a multiple photon emitting radioisotope, and the detecting comprises multiphoton detection.

36. The method of claim 35, wherein the radioisotope is an electron capture isotope of Re, Os, Ir, Pt, or Au.

37. The method of claim 26, wherein ionization produces a ratio of molecules carrying a single charge to multiple charges of greater than 9:1.

38. A device for sequencing DNA comprising:
means for producing four populations of pluralities of duplex nucleic acids, each nucleic acid having a common end and a terminal base at the other end, and a length corresponding to the position of the terminal base in the nucleic acid of interest, the duplex nucleic acids having an ionization target, and a detection label associated with the termination base,
means for ionizing the ionizing targets of the populations of duplex nucleic acid with an ionizing agent,
means for fractionating the populations of duplex nucleic acid using mass spectroscopy,
means for detecting the detection label on the terminal base of each duplex nucleic acid, and
means for determining the sequence length based on mass.

39. A population of duplex DNA molecules of lengths greater than about 50 bases, corresponding to the sequence of a nucleic acid of interest, each molecule having a common end and a terminal base at the other end, and a length corresponding to the position of the terminal base in the nucleic acid of interest, and each molecule having an ionization target and a detection label associated with the terminal base, each molecule being susceptible to ionization to produce essentially a single charge state for that length.

40. The population according to claim 39, wherein the molecules of the population are mass balanced by isotopic substitution so that the mass of the A−T pairs equals that of the G−C pairs.
